(19) United States
(12) Patent Application Publication
(10) Pub. No.: US 2003/0108514 A1
(43) Pub. Date: Jun. 12, 2003

(54) CHEMOKINES AS ADJUVANTS

(76) Inventor: JAMES LILLARD, BIRMINGHAM, AL (US)

Correspondence Address: GLENN HENDRICKS, PO BOX 2509, FAIRFAX, VA, 220312509

(21) Appl. No.: 09/212,600

(22) Filed: Dec. 16, 1998

Related U.S. Application Data
(60) Provisional application No. 60/069,843, filed on Dec. 17, 1997.

(51) Int. Cl. A61K 38/19; A61K 39/38; A61K 39/00

(52) U.S. Cl. 424/85.1; 424/184.1

(57) ABSTRACT

This invention provides means of enhancing immune response, particularly mucosal immune response, by administration of an immune-enhancing effective amount of an epithelial growth factor, CC or C×C a chemokine in a pharmaceutically acceptable carrier. Epithelial growth factor and CC or C×C chemokines may be delivered to the mucosa in conjunction with antigen to provide improved cell-mediated immune response to the antigen. Mucosal means of application include oral, intranasal, ocular, intravaginal, rectal and/or intraurethral administration in liquid or particulate form. The adjuvants may, additionally, be added to liquids or solids for administration. For example, the adjuvants of the invention may be administered in feed or water or on solid supports such as sponges and fabrics. The vaccines with the adjuvants may be administered in or on baits.

This application takes priority from U.S. Provisional Patent Application No. 60/069,843 filed Dec. 17, 1997.

### FIELD OF THE INVENTION

This invention relates to use of mitogenic and chemokinetic factors such as epithelial growth factors, CC and C×C chemokines for use as adjuvants to boost immune response.

### BACKGROUND OF THE INVENTION

The immune response mechanism involves both systemic and localized mucosal responses to pathogens and to vaccines. The response to the immunogen or pathogen may be cell-mediated or humoral. (See Fundamental Immunology, 3rd. edition, (W. E. Paul, Editor), Raven Press, New York, (1993).) For example, many intestinal pathogens require a mucosal immune response to provide effective protection from illness.

The use of an adjuvant as a means of enhancing responses to immunogens has long been known. Adjuvants may function in several ways. Some act on the immune system to elicit a more effective antibody reaction to the antigen by activating host macrophages, dendritic cells, B cells and T cells, or by enhancing antigen presentation. Adjuvants may enhance immune responses by prolonging the release of antigen, increasing antigen uptake, up-regulating antigen processing, stimulating cytokine release, stimulating B cell switching and maturation and/or eliminating immuno-suppressor cells. Presently known adjuvants include aluminum hydroxide and Freund's complete adjuvant. These adjuvants are parenterally administered and fail to induce local immune responses. A list of the most effective adjuvants would include bacterial toxins which may be administered with the target immunogen. Sometimes these immune response-enhancing molecules are bound to the toxin. While these adjuvants induce local immune responses, many of these adjuvants cause serious untoward effects.

U.S. Pat. No. 5,571,515 discloses the use of IL-12 as an adjuvant for use to enhance cell-mediated immunity.

U.S. Pat. No. 4,180,563 teaches immunostimulants from bacterial cells. The document suggests that immunostimulant agents may be obtainable from cells of animal origin, but suggests that such immunostimulants may arise because of activity of bacteria in the tissues.

WO 97/41831 teaches use of various cytokines to “activate, stimulate and/or inhibit their immune system”. The narrative generally refers to IL-2, IL-12, IL-15, IFN-α, IFN-γ IFN-β, and the CD-40 ligand. That reference teaches “the present immunotherapy may be extended for about one or two years, even longer periods, or be administered continuously as a maintenance therapy, for example, to those individuals suffering from a congenital or acquired immunodeficiency, chronic inflammation, etc.” While use of all cytokines as a class as adjuvants is suggested, the cytokines exemplified therein are not appropriate for use as adjuvants in the methods taught in the instant disclosure.

Epithelial cell populations of the gastrointestinal and respiratory tract have a high turnover rate due to aging or cell death and damage by toxins and pathogens. Growth factors regulate epithelial cell turnover and thus contribute to the progress and eventual outcome of disease. Growth factors are produced by endothelial cells, keratinocytes, epithelial and immune cells. These molecules bind seven-transmembrane receptors on host cells to regulate epithelial cell turnover, growth, chemotaxis and in general contribute towards the outcome from the disease process. Epithelial growth factors (EGFs) are believed to sustain the mucosa by stimulating epithelial cell proliferation and repair. It had previously been reported that these growth factors sustained the mucosa by stimulation of epithelial proliferation and associated cell repair.

Similar to EGFs, CC and C×C chemokines bind seven transmembrane receptors, are produced by a variety of cell types in response to various external stimuli such as toxins and pathogens, are mitogenic and activate many eukaryotic cell types (e.g. lymphocytes).

There are four classes of chemokines (CC, C×C, C and C×3C, which have conserved cysteine (C) residues important for their conformation. Of the C×C chemokines described herein, interferon gamma inducible protein 10 (IP-10) has been shown by others to be produced by monocytes, fibroblasts, endothelial cells and keratinocytes and is chemotactic or hapotactic for monocytes, natural killer (NK) cells and activated T cells. Pre-B-cell growth-stimulating factor/stromal cell-derived factor-1 (SDF-1) activates resting NK, T and B cells and activated lymphocytes. Growth stimulating factor or macrophage inflammatory peptide-2 (MIP-2) is produced by monocytes, fibroblasts and endothelial cells and is also chemotactic or hapotactic for B cells and neutrophils.

Some of the CC chemokines such as RANTES (regulated on activation of normal T-cell expressed and secreted chemokine) macrophage inflammatory peptide-1α (MIP-1α, also called LD-78), macrophage inflammatory peptide-1β (MIP-1β, also called ACT-2) and monocyte chemotactic protein (MCP-1) (also called, monocyte chemotactic activating factor) are also effective for use as described in this disclosure. RANTES has been shown by others to be produced by T lymphocytes and platelets and attracts monocytes, NK cells, memory T cells, eosinophils and basophils. MIP-1α and MIP-1β are expressed by T cells as well as monocytes. However, MIP-1α is chemotactic for monocytes, eosinophils, basophils, B cells, NK cells and activated CD8⁺ T cells, MIP-1β i chemotactic for monocytes, NK cells, eosinophils and activated CD4⁺ T cells. Additionally, SDF-1, RANTES and MIP-1α have been shown to be important in the pathology of human immunodeficiency virus infections.

In the adult human, the mucosal surface is greater than 300 m² and requires a significant number of lymphoid cells for its protection. Effector molecules, like secretory immunoglobulin A antibodies (S-1gA Abs), CC and C×C chemokines as well as peptide growth factors, which include EGF, transforming growth factors (TGF-α and (TGF-β), fibroblast growth factor, insulin and insulin-like growth factors, hepatocyte growth factor, and intestinal trefoil factors, are produced by cells in the mucosa. All of these factors play a role in maintaining the integrity of mucosal surfaces. S-IgA Abs in the mucosa represent one of the first lines of defense against invading pathogens or toxins that, if left untalted, lead to pathology. Unfortunately, in the context of vaccine development, attempts to induce these protective Abs have not met with great success.

### SUMMARY OF THE INVENTION

This invention provides means of enhancing immune response, particularly mucosal immune response, by administration of an immune-enhancing effective amount of an epithelial growth factor, CC or C×C a chemokine in a pharmaceutically acceptable carrier. Epithelial growth factor and CC or C×C chemokines may be delivered to the mucosa in conjunction with antigen to provide improved cell-mediated immune response to the antigen. Mucosal means of application include oral, intranasal, ocular, intravaginal, rectal and/or intraurethral administration in liquid or particulate form. The adjuvants may, additionally, be added to liquids or solids for administration. For example, the adjuvants of the invention may be administered in feed or water or on solid supports such as sponges and fabrics. The vaccines with the adjuvants may be administered in or on baits.

### DETAILED DESCRIPTION OF THE INVENTION

Epithelial growth factors, CC and C×C chemokines are generated by the epithelium of the mucosa and by lymphocytes. These agents act on the immune cells of the mucosa (e.g., upper and lower respiratory, gastrointestinal and reproductive tract). The EGF used as described herein was produced by recombinant technology and was purchased from CalBiochem, Inc., Palo Alto, Calif. The CC and C×C chemokines were produced by recombinant technology and obtained through Dennis Taub of the National Cancer Institute.

While bacterial toxins can boost S-IgA, these substrates have deleterious side effects in humans and other mammals. Fortunately, it is now possible, by using epithelial growth factors, CC and C×C chemokines in accord with the teachings herein, to induce significant and protective antigen-specific S-IgA Abs in mucosal secretions. Furthermore, the strategy disclosed herein initiates serum IgA, IgM and IgG with mixed T helper type 1 and 2 (Th1/Th2) responses. Comparative humoral and cellular immune responses have been shown to protect laboratory animals against lethal doses of mucosal and systemic pathogens and toxins.

Epithelial growth factors, CC and C×C chemokines can be used as adjuvants in systemic and local, particularly mucosal, vaccine preparations. These protein-based vaccines can facilitate mucosal and systemic immunity to immunogens when given in compositions containing in combination the adjuvants disclosed herein with the target antigen or administered as separate compositions given in conjunction with exposure to the antigen to enhance immune response to the antigen.

The best known of the effective mucosal vaccines is the Salk polio vaccine. Several antigens are also available to raise immune response to intestinal diseases such as diarrhea arising from E. coli or Shigella species. The adjuvants of the invention are also particularly valuable for use with vaccines against hepatitis and human immunodeficiency virus. In all of these and similar instances, the use of epithelial growth factors, CC and/or C×C chemokines to enhance immune response would be appropriate.

Materials and Methods:

Immunizations:

All mice used were 8 to 10 week old C57BL/6 mice (Charles River Laboratories, Willmington, Mass.) housed in laminar cabinets. The mice were free of microbial pathogens, as determined by routine histological analysis. Mice were intranasally immunized with 10 μl (5 μl per nostril) of sterile phosphate buffered saline (PBS), pH 7.5 containing 25 μg chicken egg albumin (OVA from Sigma Chemical Col, St. Louis, Mo.) alone or with 0.01, 0.1, 1.0 or 5 μg of EGF, IP-10, SDF-1, MIP-2, RANTES, MIP-1α, MIP-1β or MCP-1 on days 0, 7 and 14.

Sample Collection:

Serum samples were collected via retro-orbital puncture using sterile heparinized capillary tubes. Vaginal secretion samples were obtained by flushing the vaginal cavity with 50 μl PBS three times for a total volume equal to about 150 μl. Fecal pellets were collected, weighed and dissolved in PBS containing 0.1% sodium azide (100 mg fecal pellet per 1 ml PBS/sodium azide). These samples were vortexed, centrifuged and the supernatants were collected for analysis. These mucosal and serum samples were accumulated at weekly intervals and analyzed for antigen (e.g., OVA)-specific IgA, IgM, IgG, IgE, IgG1, IgG2a, IgG2b and IgG3 antibody titers. Mice were sacrificed on day 21 for analysis of OVA-specific Ab forming cells and T cell proliferative and cytokine profile responses.

Cell Preparation:

Submandibular and cervical lymph nodes (SM/CLN), mesenteric lymph nodes (MLN), Peyer's patches (PP), vaginal ileal lymph nodes (ILN) and spleen (SP) suspensions were made by passage of tissue through wire mesh. After the excision of PPs, the small intestine was isolated to determine the Ig secreting cells in the intestinal tract which directly relate to protection against intestinal pathogens. The intestinal tissue was then gently cleaned, minced and treated with 1 mM EDTA in PBS at 37° C. with agitation for 15 to 30 minutes. Next, these tissues were treated with collagenase in RPMI media for approximately 1 hour. Finally, lamina propria lymphocytes (LPL) were isolated using a percoll (Pharmacia, Uppsala, Sweden) gradient. The LPLs were analyzed to ascertain the antigen specific Ig secreting cells in the intestine which are essential for protection against pathogens and toxins.

The lower respiratory tract (lung) and salivary gland (SG) tissues were isolated, cleaned, minced and washed in PBS. These tissues were also digested with collagenase, isolated and examined to determine the antigen-specific Ig secreting cells and T cell-mediated immunity, which are important for lower and upper respiratory immunity.

The nasal tract and nasopharyngeal-associated lymphoid tissue (NALT) was isolated and passed over sterile glass fiber to acquire a single cell suspension of lymphocytes. The nasal tract and NALT were studied to determine the number of Ig secreting cells in the upper respiratory tract needed for protection against respiratory pathogens and toxins.

Antigen-specific Antibody Titer Detection by ELISA and ELISPOT Assays:

Antibody titers in sera and secretions were analyzed by ELISA to confirm the source of antigen-specific antibodies detected by ELISA, quantitation of vaccine antigen-specific antibody spot forming cells from the SP, PP, MLN, SM/CLN, lung and NALT were enumerated by ELISPOT analysis.

Enumeration of Antigen-Specific T Cell Proliferative Responses:

T cell depleted irradiated (3,000 rads) spleen cells from naive mice were used as feeder cells for T cell proliferation assays. T cells from the SM/CLN, MLN, PP, lung, ILN and SP of immunized mice were purified using a nylon wool column. Purified T cells (2.5×10⁵ cells/ml) were cultured with or without 0.5 mg/ml OVA plus feeder cells (0.5×10⁶ cells/ml) in complete RPMI media in round bottom tissue culture treated 96-well plates. Cells were incubated at 37° in 5% CO₂. After 48 hours of incubation, 10 μl of 50 μCi/ml [methyl-³H]-thymidine was added to each well. Proliferation or thymidine uptake was measured 18 hours later. The stimulation index of the various samples was determined and expressed as the counts per minutes (CPM) of cultures containing OVA divided by the CPMs of cultures lacking OVA.

CD4⁺ T cells that had been isolated using a mouse CD4 isolation column were cultured with antigen as above. Cultured supernatants were harvested after 5 days of incubation for cytokine quantitation.

Results:

The administration of EGF, IP-10, SDF-1, MIP-2, RANTES, MIP-1α, MIP-1β or MCP-1 in conjunction with vaccine resulted in increased IgA, IgG, (IgG1, IgG2a, IgG2b, IgG3) and IgM titers in the serum. Increase in fecal IgA and IgG and vaginal IgA and IgG was also found. Antibody spot forming cells from NALT, SP, PP, MLN, lung and SM/CLN were shown to secrete antigen-specific IgA, IgM and IgG antibodies. Additionally, augmentation of antigen-specific T cell proliferation of immunized mice was also observed in lymphocytes isolated from SM/CLN, MLN, PP, spleen, lung and ILN. Vaccinated mice also displayed higher antigen-specific Th1 and Th2 type cytokine responses. Hence, it can be seen that the immune responses were increased by exposure of the mucosa to these adjuvants.

Compositions of epithelial growth factors, CC or C×C chemokines in cellular immune enhancing amounts may advantageously be administered at very low levels in conjunction with vaccines. For example, dosages such as 1 to 10 ηg in small animals and from 10 μg to 10 mg epithelial growth factors, CC or C×C chemokines in large mammals may be administered. These agents may be administered in the usual pharmaceutical carriers such as saline, buffered saline, glucose, etc. Preferred methods of administration involve direct application to the mucus membranes. Such compositions may, for example, be provided in the form of drops, such as nose, ear or eye drops or in sprays. Dry preparations such as lyophilized epithelial growth factors, CC or C×C chemokines with powdered carriers may, for example, be inhaled or sprayed on the mucosa. Such compositions may also be provided in capsules or in tablet form for ingestion. The epithelial growth factors, CC or C×C chemokines may also be administered on a solid support such as a sponge or fiber material. For example, such supports with vaccine and the inventive adjuvants may be applied to abraded skin. Such administration is particularly valuable for use in environments where access to sterile equipment is limited. Compositions for oral ingestion may be enteric coated. The adjuvants may, additionally, be added to liquids or solids for administration by mouth. For example, the adjuvants of the invention may be administered in feed or water or on solid supports such as sponges and fabrics. For example, the adjuvants may be administered in or on baits.

The adjuvants may be given orally in alkaline solutions containing antigens appropriate for raising antibodies against organisms which give rise to intestinal diseases to raise mucosal antibodies. Alkaline solutions such as those containing bicarbonates protect antigens and adjuvants from destruction in the upper GI tract.

What is claimed is:

1. A method of enhancing immune response in a mammal by administration of an immune-enhancing effective amount of at least one active agent chosen from epithelial growth factors, CC and C×C chemokine which gives rise to increased IgA, IgG and IgM titers in a pharmaceutically acceptable carrier.

2. A method of claim 1 wherein the epithelial growth factors. CC or C×C chemokine is administered directly to the mucosa.

3. A method of claim 2 wherein the epithelial growth factor, CC or C×C chemokine is administered in the form of drops or a spray.

4. A method of claim 2 wherein the epithelial growth factor, CC or C×C chemokine is administered in powder form.

5. A method of claim 1 wherein the epithelial growth factor, CC or C×C chemokine is on a solid support.

6. A method of claim 1 wherein the epithelial growth factor, CC or C×C chemokine is administered orally.

7. A method of claim 1 wherein the active agent is EGF.

8. A method of enhancing the immune response by administration of at least one agent chosen from among epithelial growth factors, CC and C×C chemokines in water or feed.

9. A method of claim 8 wherein the epithelial growth factor, CC or C×C chemokine is administered in animal feed.

10. A composition of matter comprising an immune-enhancing effective amount of at least one active agent chosen from epithelial growth factors, CC and C×C chemokine which gives rise to increased IgA, IgG and IgM titers and at least one immunogen which gives rise to mucosal immunity, in a pharmaceutically acceptable carrier.

11. A composition of claim 10 which has a pH greater than 7.5.

12. A composition of claim 10 in powder form.

13. A composition of claim 10 on a solid support.

14. A composition of claim 10 containing EGF.

15. A composition of claim 10 in the form of a liquid.

16. A composition of claim 10 in powder form.
